(19) United States
(12) Patent Application Publication
(10) Pub. No.: US 2007/0016061 A1
(43) Pub. Date: Jan. 18, 2007

(54) System for measuring and tracking human body fat

(75) Inventors: Luiz B. Da Silva, Danville, CA (US); George Choi, Redwood City, CA (US)

Correspondence Address: John P. Wooldridge, Esq., 114 Honu'ea Pl., Kihei, HI, 96753, US

(73) Assignee: IntelaMetrix, Inc.

(21) Appl. No.: 11/415,560

(22) Filed: May 1, 2006

Related U.S. Application Data
(63) Continuation-in-part of application No. 11/302,039, filed on Dec. 12, 2005.
(60) Provisional application No. 60/676,325, filed on Apr. 30, 2005.
(60) Provisional application No. 60/634,911, filed on Dec. 10, 2004.

(51) Int. Cl. A61B 8/14

(52) U.S. Cl. 600/459

(57) ABSTRACT

A system for evaluating health, wellness and fitness, and in particular, to a system that uses an ultrasound transducer to accurately measure fat thickness at a plurality of sites on the human body, records these measurements for long term monitoring, and based on the plurality of measurements calculates the total body composition. The system includes a central control unit to analyze the measurement and display the results in a variety of formats.

This application claims priority to U.S. Provisional Patent Application Ser. No. 60/676,325, filed Apr. 30, 2005, titled: “System for Measuring and Tracking Human Body Fat”, incorporated herein by reference. This is a continuation-in-part of U.S. patent application Ser. No. 11/302,039, filed Dec. 12, 2005, titled: “Tissue Thickness Measurement Device”, incorporated herein by reference, which claims priority to U.S. Provisional Patent Application Ser. No. 60/634,911, titled: “Tissue Thickness Measurement Device,” filed Dec. 10, 2004, incorporated herein by reference.

### BACKGROUND OF THE INVENTION

1. Field of the Invention

The present invention generally relates to the fields of fitness, and healthcare, and more particularly, it relates to systems that measure and record fat thickness at a plurality of sites on the human body.

2. Description of Related Art

Knowledge of the thickness of tissue layers, and in particular adipose (fat) and muscle tissue, can be important in the evaluation of the fitness and health of an individual. There are a variety of techniques currently used to measure the thickness of the adipose layer. For example skin calipers can be used to measure the thickness of the skin fold produced when the operator pinches a subject's skin. Various equations are used to predict body density and the percent of body adipose tissue (American College of Sports Medicine (ACSM) “Guidelines For Exercise Testing And Prescription”, 53-63 (1995)). However, there are many drawbacks to this form of adipose tissue measurement These measurements are heavily dependent on the operator, and errors and variations frequently occur. Skin fold calipers can only provide an estimate of tissue thickness and are not particularly accurate for tracking small changes.

Another means of determining body density and estimating percent body adipose tissue is a generalized measurement called hydrostatic weighing. Hydrostatic weighing requires the subject to be completely immersed in water. This method of measurement is often impractical and costly.

There is a need for an accurate, convenient, cost effective system for measuring and monitoring human body fat The present invention fulfills this need, and further provides related advantages.

### SUMMARY OF THE INVENTION

It is an object of the present invention is to provide a system for measuring, analyzing, and recording human body fat thickness.

Another object of the present invention is to provide a system that can provide information about the health and fitness of a user.

These and other objects will be apparent to those skilled in the art based on the teachings herein.

The present invention uses ultrasound in a hand held device that connects either through a cable (e.g., USB) or wireless technology (e.g., Bluetooth) to a computer that collects and analyzes the measurements to provide the user with information related to health and fitness. The data can be recorded to allow the user to track changes and monitor trends in their health and fitness. The application software can also analyze the recorded data to provide the user with recommendations and health risk

Other objects and advantages of the present invention will become apparent from the following description and accompanying drawing

### BRIEF DESCRIPTION OF THE DRAWINGS

The accompanying drawings, which are incorporated into and form part of this disclosure, illustrate embodiments of the invention and together with the description, serve to explain the principles of the invention.

FIG. 1 is an illustration of an embodiment of the present invention for measuring body fat

FIG. 2 shows a plot of the measured ultrasound signal on the abdomen of a male.

FIG. 3 shows a plot of the measured ultrasound signal on the bicep of a male.

FIG. 4 shows a prototype of the present invention.

FIG. 5 shows the opening screen.

FIG. 6 shows the Create New Client's Profile screen.

FIG. 7 shows the Open Existing Client screen.

FIG. 8 shows the Body View screen for males.

FIG. 9 shows the Body View screen for females.

FIG. 10 shows the Measure screen.

FIG. 11 shows a signal that displays a clear boundary between fat and muscle at approximately 14 mm.

FIG. 12 shows the My Health screen.

FIG. 13 shows the Trends screen.

### DETAILED DESCRIPTION OF THE INVENTION

The present invention relates to a system for evaluating health, wellness and fitness, and in particular, to a system that uses an ultrasound transducer to accurately measure fat thickness at a plurality of sites on the human body, records these measurements for long term monitoring, and based on the plurality of measurements, calculates the total body composition.

In one embodiment, the present invention comprises a hand held ultrasound transducer that attaches through a cable (e.g., USB) or wireless connection (e.g., Bluetooth) to a computer that includes a software program that collects the recorded ultrasound signal The software program analyzes the signal from each measurement point on the body and, using a minimum of one point, calculates the estimated total body fat The program can also use multiple measurement points to increase total accuracy of the body fat measurement Measured body fat percentage is used by the program to advise the user of fitness and relative risk of disease. Changes in the percentage of body fat are used to show the user the resulting modifications to the body shape.

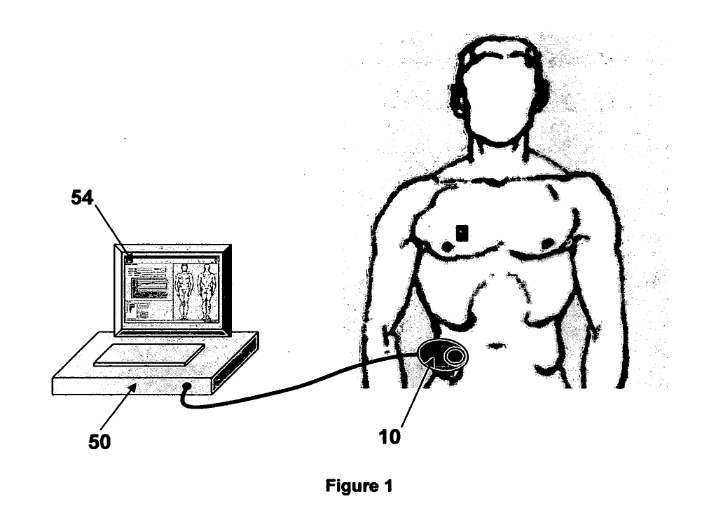

FIG. 1 illustrates how the present invention can be used to measure the local tissue structure. The measuring device 10 is placed on the skin at a point of interest When activated, an ultrasound signal is transmitted into the tissue and the return signal is collected. The collected signal is then communicated by cable or by wireless means to the remote control unit 50. The control unit 50 displays the recorded waveforms and the calculated thickness of relevant layers on a monitor 54. In addition, the control unit 50 stores the waveforms and information about the location of the measurement so that the user can easily monitor changes over time. The control unit can be a portable computer, or PDA (e.g., HP Ipaq, Palm Pilot, etc.). In another embodiment, the device 10 is self contained and a small LCD display on the device 10 displays a summary of each measurement

For the present invention, the operating frequency of the transducer will typically be in the range of 500 kHz to 10 MHz. The higher frequencies have higher spatial resolution but suffer from high tissue attenuation, which limits the thickness of tissue that can be measured. In addition, it is sometimes beneficial to operate the ultrasound transducer at two different frequencies. Since the scattered signal scales strongly with the ultrasound wavelength, the ratio of scattered signal at two frequencies can be used to determined tissue properties.

A curved transducer may be used to provide a weakly focused beam that measures properties over a less than 5 mm diameter region. A small diameter reduces the blurring of layer boundaries due to non-planar layer contours. The transducer is used to both generate the ultrasound pulse and measure the time history of the return acoustic signal. The collected time history signal is a measurement of the back-scattered signal as a function of depth averaged over the ultrasound beam area. The control electronics collect and digitize the signal for further display and analysis. For additional information on transducer design and operation refer to “The Physics of Medical Imaging” Ed. Steve Webb (1988), incorporated herein by reference, and “Ultrasound in Medicine” Ed. F. A. Duck, A. C. Baker, H. C. Starritt (1997), incorporated herein by reference. See also U.S. Pat. No. 5,699,806, titled: “Ultrasound System With Nonuniform Rotation Corrector”, incorporated herein by reference.

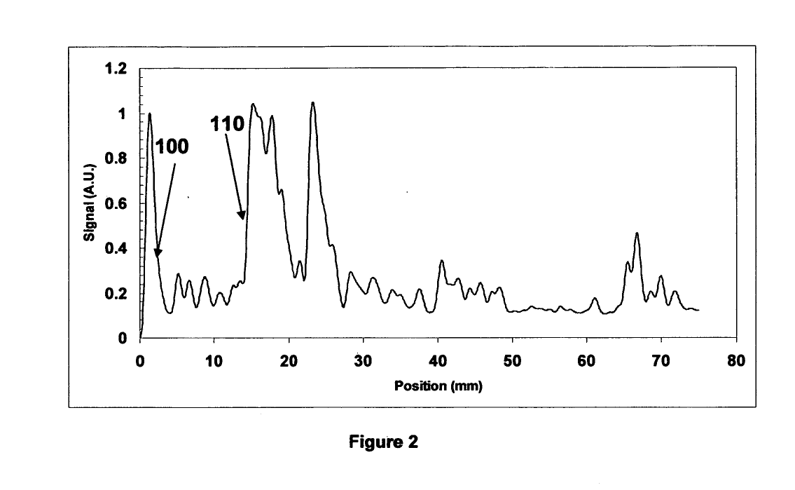

FIG. 2 shows a measured signal using the present invention on a male abdomen. The signal peaks correspond to the interface between the device and skin 100 and fat and muscle 110. The adipose (fat) layer is located between 100 and 110 and is approximately 9.8 mm thick. Strong ultrasound reflections occur at the interfaces due to impedance mismatch between the various materials. The time history is converted to thickness by the software by using average sound speeds (c). For example, c˜1600 m/s for skin, 1400 m/s for fat, 1600 m/s for muscle, and 3500 m/s for bone (See “Ultrasound in Medicine” Ed. F. A Duck, A. C. Baker, H. C. Starritt).

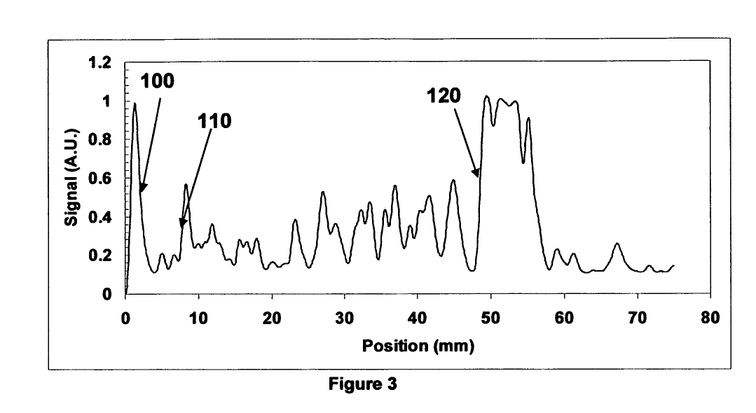

FIG. 3 shows a measured signal using the present invention on a male bicep muscle. The signal peaks correspond to the interface between the device and skin 100 and fat and muscle 110 and muscle and bone 120. The adipose layer is located between 100 and 110 and is approximately 3.2 mm thick. The muscle layer is located between 110 and 120 and is approximately 40.8 mm thick.

In normal use the measuring device would be applied at a single point or multiple key anatomical points. By making measurements at multiple sites (at least three) you can estimate the body density (D) and the percentage body fat (% BF). The most common sites used for these estimates are:

| | |
|---|---|
| TRICEPS | At the level of the mid-point between acromial process |
| | (boney tip of shoulder) and proximal end of the radius bone |
| | (elbow joint), on the posterior (back) surface of the arm. |
| BICEPS | The same level as for triceps, though on the anterior (front) |
| | surface of arm. |
| SUBSCAPULA | 2 cm below the lower angle of the scapula (bottom point of |
| | shoulder blade) on a line running laterally (away from the |
| | body) and downwards (at about 45 degrees). The fold is lifted |
| | in this direction. |
| AXILLA | The intersection of a horizontal line level with the bottom |
| | edge of the xiphoid process (lowest point of the breast bone), |
| | and a vertical line from the mid axilla (middle of armpit). |
| ILIAC CREST | The site immediately above the iliac crest (top of hip bone), at |
| | the mid-axillary line. |
| SUPRASPINALE | The intersection of a line joining the spinale (front part of iliac |
| | crest) and the anterior (front) part of the axilla (armpit), and a |
| | horizontal line at the level of the iliac crest. |
| ABDOMINAL | 5 cm adjacent to the umbilicus (belly-button). |
| FRONT THIGH | The mid-point of the anterior surface of the thigh, midway |
| | between patella (knee cap) and inguinal fold (crease at top of |
| | thigh). |
| MEDIAL CALF | The point of largest circumference on medial (inside) surface of the calf. |
| CHEST | Between the axilla and nipple as high as possible on the |
| | anterior axillary fold (males only). |

For example, by taking measurements at chest, abdomen, and thigh you can estimate the body density (D) and percentage body fat (% BF) with the following equations for males and females respectively.

For Males: D=1.10938−(0.0008267×sum of chest, abdominal, thigh)+(0.0000016×square of the sum of chest, abdominal, thigh)−(0.0002574×age). Equation is based on a sample of males aged 18-61 Jackson, A. S. & Pollock, M. L. (1978) “Generalized equations for predicting body density of men”, British J of Nutrition, 40: p497-504.).

D=1.1043−(0.001327×thigh)−(0.00131×subscapular), based on a sample aged 18-26. Sloan AW: “Estimation of body fat in young men”, J Appl. Physiol. (1967);23:p311-315.

% BF=(0.1051×sum of triceps, subscapular, supraspinale, abdominal, thigh, calf)+2.585, based on a sample of college students. Yuhasz, M. S.: Physical Fitness Manual, London Ontario, University of Western Ontario, (1974).

For Females: D=1.0994921−(0.0009929×sum of triceps, suprailiac, thigh)+(0.0000023×square of the sum of triceps, suprailiac, thigh)−(0.0001392×age), based on a sample aged 18-55. Jackson, et al. (1980) “Generalized equations for predicting body density of women”, Medicine and Science in Sports and Exercise, 12:p175-182.

D=1.0764−(0.0008×iliac crest)−(0.00088×tricep), based on a sample aged 17-25. Sloan, A. W., Burt A. J., Blyth C. S.: “Estimating body fat in young women”, J. Appl. Physiol. (1962);17:p967-970.

% BF=(0.1548×sum of triceps, subscpular, supraspinale, abdominal, thigh, calf)+3.580, based on a sample of college students. Yuhasz, M. S.: Physical Fitness Manual, London Ontario, University of Western Ontario, (1974).

Although these equations refer to thickness measurements taken with calipers, they can also be applied when fat thickness measurements are made with the more accurate device disclosed herein. In addition, a wide variety of other equations exist that offer greater accuracy; however, some require additional information (e.g., accurate age, body type).

Software within the control unit can guide the user through the process of collecting measurements at the key anatomical sites and then display the calculated % body fat (% BF) and Body Density (D).

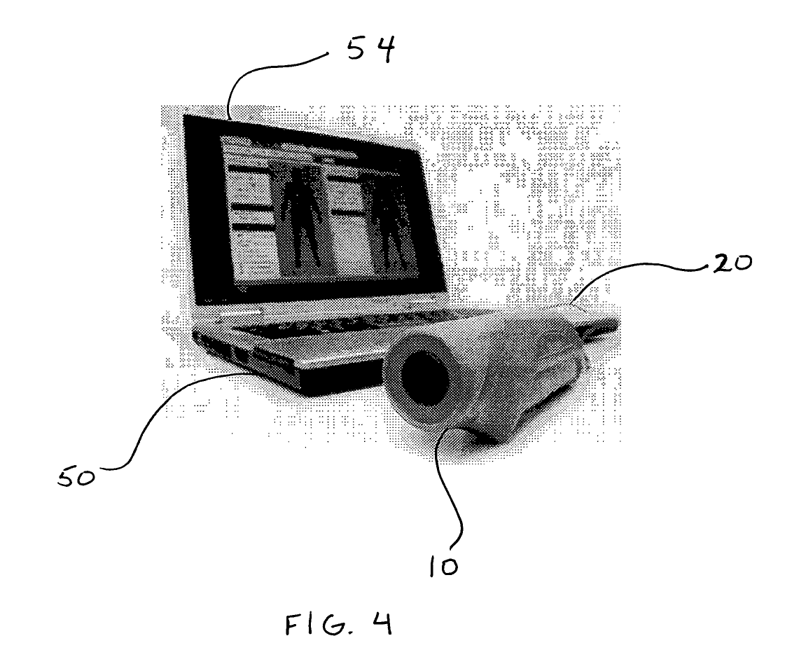

FIG. 4 shows a prototype of the present invention. A handheld ultrasound transducer 10 connected via an USB cable 20 to a laptop computer 50 running the body composition analysis software.

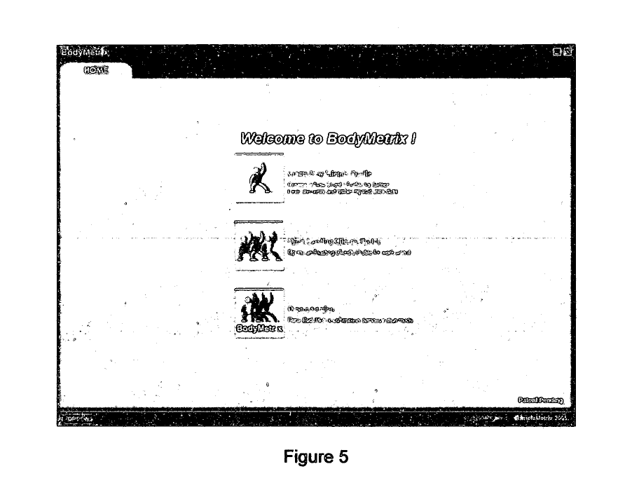

The software program “Body Metrix” controls the ultrasound measurement device and provides the user with a wide variety of tools, including body morphing, fat thickness measurement, total body fat percentage measurement, and health risk analysis. The program can run on a desktop computer, portable computer, or PDA device (e.g., HP IPAQ). The features and a sample of the screens displayed by the program are shown in the following pages and further explained at the Intela Metrix website located at www. intelametrix.com/ incorporated herein by reference and further explained in the Body View Software User Guide incorporated herein by reference and shown T www.intelametrix.com/support/Body View User Manual.pdf, incorporated herein by reference. The software contained in the produce currently on the market and publicly available is incorporated herein by reference. The opening screen is shown in FIG. 5.

The Home Screen allows the user to create a new client (or user), open the existing client data base or operate in a Demonstration mode where no data is recorded. Using option buttons the units of measure can be set to inches and pounds or centimeters and kilograms.

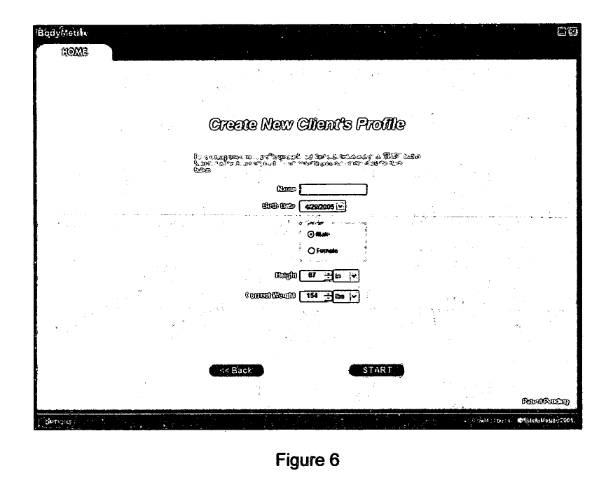

From the Home Screen the user can select to create a new client's profile. The Create New Client's Profile screen (shown in FIG. 6) allows entry of the client's name, birth date, height and weight

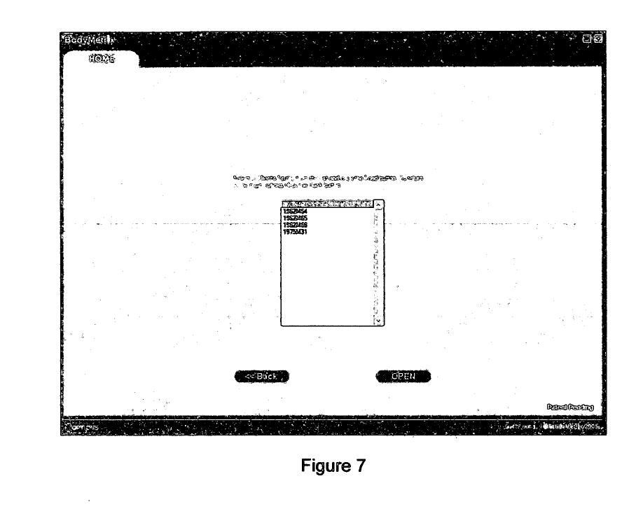

Also, from the Home Screen the user can open the existing client data base. The Open Existing Client screen (shown in FIG. 7) allows the user to retrieve previous measurements from the data base and look for trends.

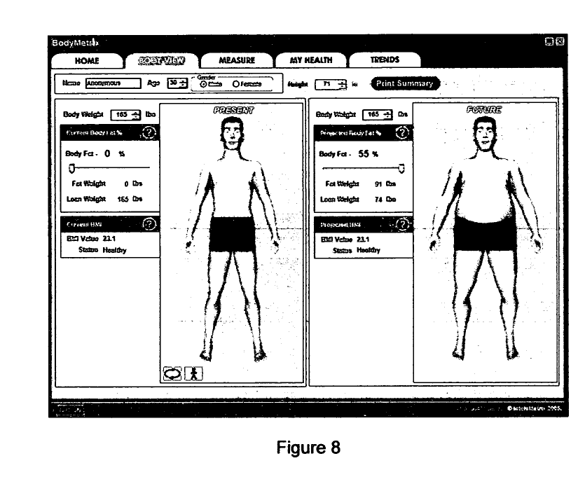

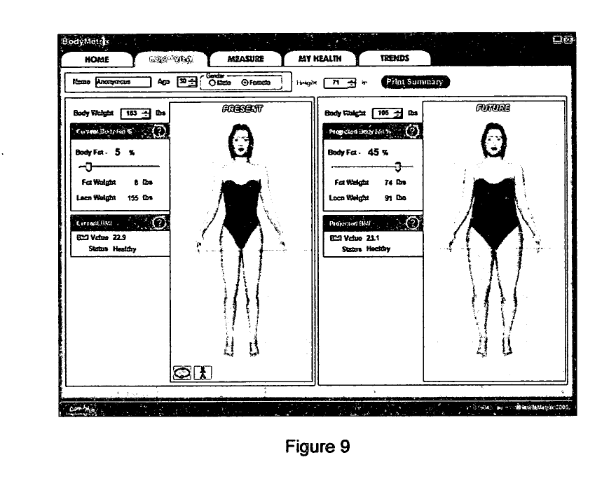

The Body View screen (as shown in FIG. 8 for male and FIG. 9 for female) allows a client to adjust the percentage of body fat to get an approximate idea of how their body shape might change. The figures can be rotated to allow a view from all angles.

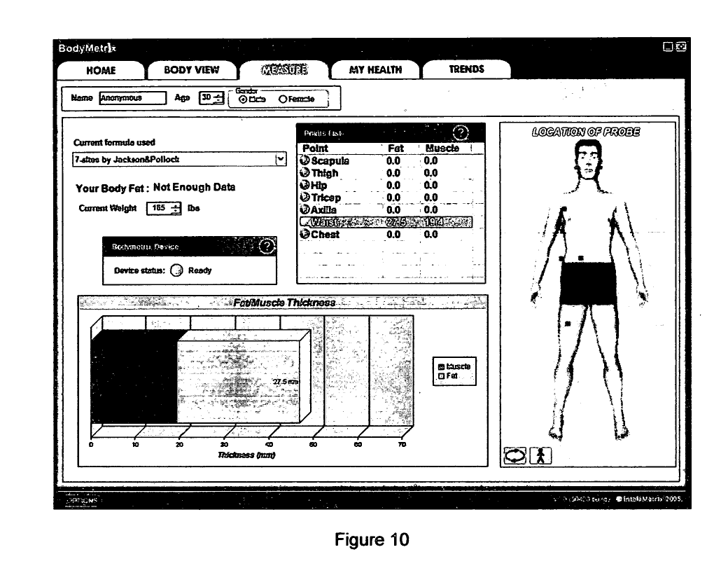

The Measure screen (FIG. 10) is used to control the measurement of fat thickness with the ultrasound transducer. From the Measure screen the user may select from a drop down menu a formula to calculate Body Fat The formulas used are those known and accepted in the health and fitness fields (e.g., 2-site Sloan, 3-site and 7-site by Jackson & Pollock). When a measurement point is selected, the location on the pictured body is marked with a red cube. The other measurement points are marked with blue cubes. The user may add points by simply moving the cursor over the body picture and clicking on the desired locations. This feature allows a client to track the fat thickness in specific points of interest

All measurements are taken from the Measure screen. To take a measurement, the user places the ultrasound device on the desired body point and presses the measure button, holding it down for approximately 1 second. When the button is released, the signal is analyzed and the estimated fat thickness and muscles thickness is displayed. This value is stored in the point list, and the user can move to the next measurement point When all desired points are measured and recorded the body fat percentage is calculated and displayed.

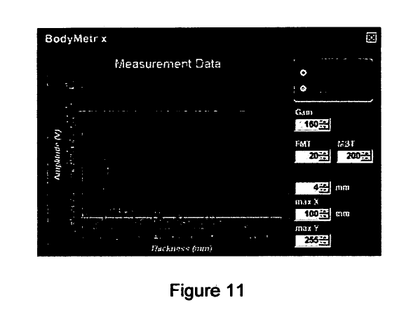

The signal displayed in FIG. 11 shows a clear boundary between fat and muscle at approximately 14 mm. This is an example of the ultrasound measurement for a specific body point (male abdomen).

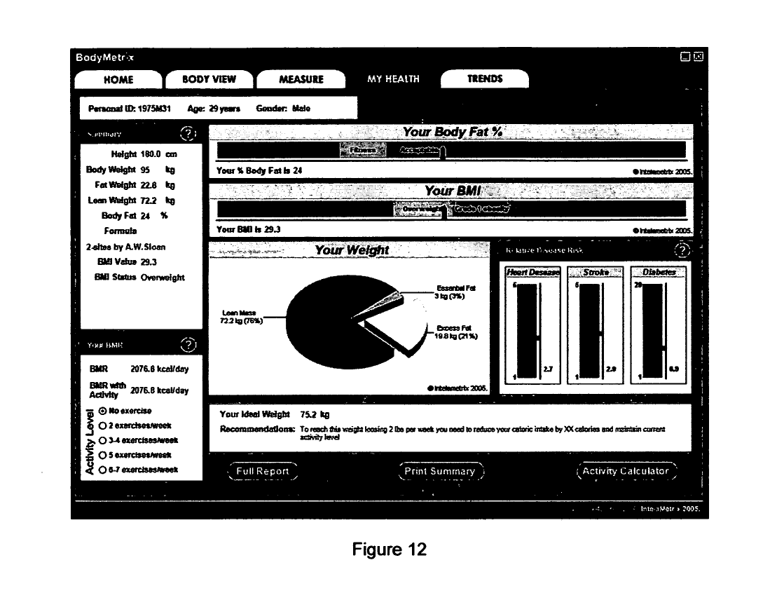

The My Health screen (FIG. 12) provides a summary of the user's present condition. This screen analyzes the information provided to give an overall picture of the user's total body composition and relative health risks. This information is provided as guidance. The user can print out a full report by clicking on the “Full Report” button or just the summary by clicking on the “Print Summary” button at the bottom of the page. The “Activity Calculator” button allows the user to calculate the number of calories burned by performing selected activities.

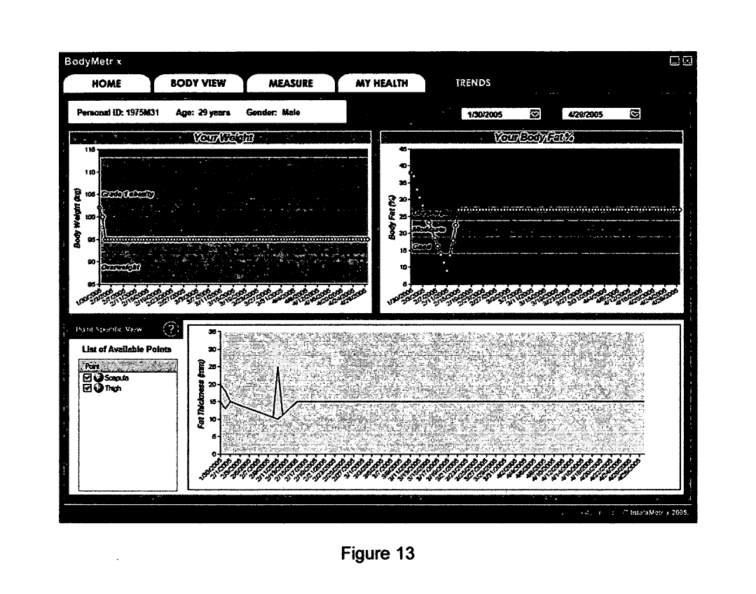

The Trends screen shown in FIG. 13 tracks a user's body composition over time. The Trends screen allows the user to monitor the changes or trends in BMI Body Fat percentage or fat thickness at selected points.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

The foregoing description of preferred embodiments of the invention is presented for purposes of illustration and description and is not intended to be exhaustive or to limit the invention to the precise form disclosed. Many modifications and variations are possible in light of the above teaching. The embodiments were chosen and described to best explain the principles of the invention and its practical application to thereby enable others skilled in the art to best use the invention in various embodiments and with various modifications suited to the particular use contemplated.

We claim:

1. An apparatus, comprising a computer system having hardware and software, wherein said software comprises means for calculating from an ultrasound return signal the location of at least one tissue boundary by using at least one parameter that is specific to a subject under test, wherein said parameter is selected from the group consisting of age, height, weight, sex, and location of said skin portion.

2. The apparatus of claim 1, wherein said computer system is operatively connect to:
an ultrasound transmitter and receiver in a handholdable housing, wherein ultrasound pulses from said transducer can be transmitted into a skin portion of a subject under test, wherein interfaces between layers beneath said skin portion will reflect a portion of said ultrasound pulses to produce a return signal, wherein said receiver can detect said return signal;
means for powering said ultrasound transmitter and receiver; and
means for transmitting signals from said ultrasound transmitter and receiver to said computer system.

3. The apparatus of claim 2, wherein said ultrasound transmitter and receiver are a single element.

4. The apparatus of claim 2, wherein said ultrasound transmitter and said receiver are two separate elements.

5. The apparatus of claim 2, further comprising means for coupling said ultrasound transmitter and receiver to said skin portion.

6. The apparatus of claim 5, wherein said means for coupling comprises a disposable ultrasound coupling gel holder.

7. The apparatus of claim 5, wherein said means for coupling comprises a refillable water compartment.

8. The apparatus of claim 7, wherein said ultrasound transducer comprises a hydrophilic surface.

9. The apparatus of claim 2, further comprising a ruler integrated onto said handholdable housing.

10. The apparatus of claim 2, further comprising an level integrated onto said handholdable housing.

11. The apparatus of claim 2, wherein said transducer comprises a curved surface configured to provide a weakly focused beam.

12. A method, comprising calculating from an ultrasound return signal the location of at least one tissue boundary by using at least one parameter that is specific to a subject under test, wherein said parameter is selected from the group consisting of age, height, weight, sex, and location of said skin portion

13. The method of claim 12, further comprising:
applying at least one ultrasound transducer to the surface of a skin portion of a subject under test;
transmitting ultrasound pulses from said transducer into said skin portion, wherein interfaces between layers beneath said skin portion will reflect a portion of said ultrasound pulses to produce a return signal; and
detecting said return signal.

14. The method of claim 12, wherein said at least one tissue boundary comprises an interface between adipose tissue and muscle.

15. The method of claim 12, wherein said at least one tissue boundary comprises an interface between muscle and bone.

16. The method of claim 12, wherein said return signal is further analyzed to determine the thickness of at least one tissue layer beneath said skin portion.

17. The method of claim 16, wherein said at least one tissue layer comprises a fat layer, wherein the steps of applying, transmitting, detecting and calculating are repeated at different locations on said subject under test to produce a plurality of return signals, the method further comprising calculating a percentage of body fat of said subject under test by using said plurality of return signals.

18. The method of claim 17, further comprising producing a map of fat thickness.

19. The method of claim 12, further comprising calculating the body mass index of said subject from the weight and height of said subject.

20. The method of claim 19, wherein said return signal is further analyzed to determine the thickness of at least one tissue layer beneath said skin portion, wherein said at least one tissue layer comprises a fat layer, wherein the steps of applying transmitting, detecting and calculating are repeated at different locations on said subject under test to produce a plurality of return signals, the method further comprising calculating a percentage of body fat of said subject under test by using said plurality of return signals, the method further comprising approximating adipose tissue thickness by relating said percentage body fat to said body mass index.
